(19) United States
(12) Patent Application Publication
(10) Pub. No.: US 2012/0265173 A1
(43) Pub. Date: Oct. 18, 2012

(54) MEDICAL DEVICE CONTAINING CATHETER ANCHORING FEATURE

(75) Inventors: George BOURNE, Southboro, MA (US); Raymond Lareau, Westford, MA (US)

(73) Assignee: NAVILYST MEDICAL, INC., Marlborough, MA (US)

(21) Appl. No.: 13/531,173

(22) Filed: Jun. 22, 2012

Related U.S. Application Data
(62) Division of application No. 12/550,317, filed on Aug. 28, 2009, now abandoned.

(51) Int. Cl. A61M 27/00

(52) U.S. Cl. 604/540

(57) ABSTRACT

An indwelling drainage catheter is disclosed that is configured to include spiral, helical or radial geometry on the external surface that allows the catheter to be introduced and locked into the anatomy via threading, linear indexing or similar action.

### CROSS REFERENCE TO RELATED APPLICATIONS

This is a divisional application of U.S. patent application Ser. No. 12/550,317, filed Aug. 28, 2009, entitled, “Medical Device Containing Catheter Anchoring Feature”, which application is incorporated by reference in its entirety.

### FIELD

The present invention relates generally to indwelling drainage catheters, and more particularly, to a catheter that is configured to include spiral, helical or radial geometry on the external surface that allows the catheter to be introduced and locked into the anatomy via threading, linear indexing or similar action.

### BACKGROUND

Flexible catheters are used for percutaneous drainage of an abscess or pocket of fluid in the body to the exterior by means of gravity or negative pressure. Fluid collection may be the result of an infection, surgery, trauma or other causes. Typical fluids include biliary, nephrostomy, pleural, urinary, and mediastinal collections. As an alternative to providing drainage, these catheters can also be used to introduce substances, such as fluids, into a patient's body.

In percutaneous drainage procedures, a catheter is typically introduced into a patient through a hypodermic needle or a trocar. A guidewire is inserted through the needle or the trocar, which is then removed. The catheter tube, with a stiffening cannula, then passes over the previously emplaced guide wire into the drainage site in the body cavity. The stiffening cannula is then removed.

Once a drainage catheter is in position in the body cavity, it is desirable to anchor the catheter before drainage begins. Typically, this can be done by forming a restraining portion in the distal end of the catheter in the form of a pigtail or “J-curve.” For a pigtail configuration, a flexible tension member, such as a suture thread, extends through draw ports at two spaced positions along the distal portion of the catheter. The restraining portion is conventionally activated by manually pulling the suture thread so that the two draw ports move toward each other as the pigtail loop forms at the distal end of the catheter. When the suture thread is taut, it prevents the pigtail loop from straightening by holding the juxtaposed portions of the catheter together in a locked position. The restraining portion is thus in a shape capable of resisting displacement from the body cavity. Once actuated, this restraining portion prevents removal of the catheter. When the catheter is ready to be removed, the cannula is inserted through the lumen until it reaches the pigtail loop. The restraining portion at the distal end is unlocked by cutting or releasing the suture at the proximal end, where the catheter protrudes from the body. Then the stiff cannula can be advanced distally to straighten the pigtail and help remove the catheter from the patient.

A preformed curve in the shape of a malecot rib has also been used as a possible anchoring mechanism. In this configuration, longitudinal slits are located in the restraining portion of the catheter at the distal end. The rib is activated in a similar manner as the pigtail configuration by manipulating a tension member, except the restraining portion is formed in the shape of multiple wings (typically two or four) instead of a pigtail.

Successful procedures involving percutaneous drainage depend upon the initial placement of the drainage catheter and having the catheter remain in place for the duration of the treatment. Without adequate anchoring or support, catheter dislodgment may result due to body movements by the patient or under other conditions.

There are disadvantages of relying on a configuration such as the pigtail or the malecot rib as the sole anchoring mechanism. For example, the actuation of a pigtail loop may not result in a precise placement because the pigtail has some compressibility and may migrate within the body cavity, causing movement at the proximal end of the catheter near the incision area as well. Due to the uncertainty of placement, additional steps may be necessary to confirm that the restraining portion has been actuated. Another potential problem relates to the structure of the pigtail. As the anchoring mechanism preventing the inadvertent removal of the catheter, the pigtail is constantly subject to forces pulling against it. Therefore, it is possible for a pigtail restraining portion to give way and collapse on itself. Such a collapse would destabilize the location of the catheter and adversely affect drainage. Additionally, the pigtail may be difficult to form or engage in small collection pools or may float in larger collection pools.

Described herein are unique devices, systems and methods for supplementing or replacing the pigtail or malecot anchoring mechanism by using a catheter with spiral, helical or radial geometry on the external surface of the catheter.

### SUMMARY

The devices, systems and methods described herein relate to percutaneous drainage catheters and an anchoring structure or mechanism for indwelling catheters (both short and long-term) via the inclusion of spiral, helical or radial geometry on the external surface of the catheter. This feature allows for the catheter to be introduced or locked into the anatomy via threading, linear indexing or similar action. The feature can complement or replace existing anchoring means such as pigtails.

In one embodiment, the catheter includes anchoring members arranged circumferentially on a portion of the catheter shaft, comprising spiraling rims on the exterior surface near the distal end to interface with the surrounding tissue to form anchoring points. It is also contemplated that the anchoring members can be located at multiple points along the catheter, such as near both the distal end and the proximal end (i.e. at the percutaneous site). The catheter can make contact with the human tissue via threading.

In another embodiment, the catheter includes annular anchoring rings that are introduced and locked into the anatomy via linear indexing or other means.

In combination with other features described herein, an alternative embodiment may include anchoring geometries with intermittent slots or spacing to enhance flexibility of the catheter or to promote anchoring.

Of the various features described, the structures herein offer a number of advantages in their construction and ability to anchor the drainage catheter in various applications. Other systems, methods, features and advantages will be or will become apparent to one with skill in the art upon examination of the following figures and detailed description. It is intended that all such additional systems, methods, features and advantages be included within this description, be within the scope of the devices, systems and methods described herein, and be protected by the accompanying claims.

### BRIEF DESCRIPTIONS OF THE DRAWINGS

The figures provided herein are not necessarily drawn to scale, with some components and features being exaggerated for clarity. Each of the figures diagrammatically illustrates aspects of the embodiments.

FIG. 1A is a schematic view of a catheter with a “pig tail” loop configuration as an anchoring mechanism, shown before the activation of the pig tail.

FIG. 1B is a schematic view of a catheter with a “pig tail” loop configuration as an anchoring mechanism, shown after the activation of the pig tail.

FIG. 2 is a perspective view depicting an exemplary embodiment of the distal portion of a drainage catheter having a spiraling geometries on the exterior.

FIG. 3 is a perspective view depicting an exemplary embodiment of the distal portion of a drainage catheter having annular geometries on the exterior.

FIG. 4 is a perspective view depicting an exemplary embodiment of the distal portion of a drainage catheter having annular slots.

FIG. 5 is a diagrammatic representation depicting an exemplary embodiment of a set of rims constituting spiral, helical or radial anchoring geometries.

### DETAILED DESCRIPTION

The devices, systems and methods described herein can be used for introducing a percutaneous catheter into a patient and anchoring the catheter into the body of the patient to facilitate draining fluid or removing other materials from the body. Alternatively, the catheter can introduce substances, such as fluids, into the patient's body.

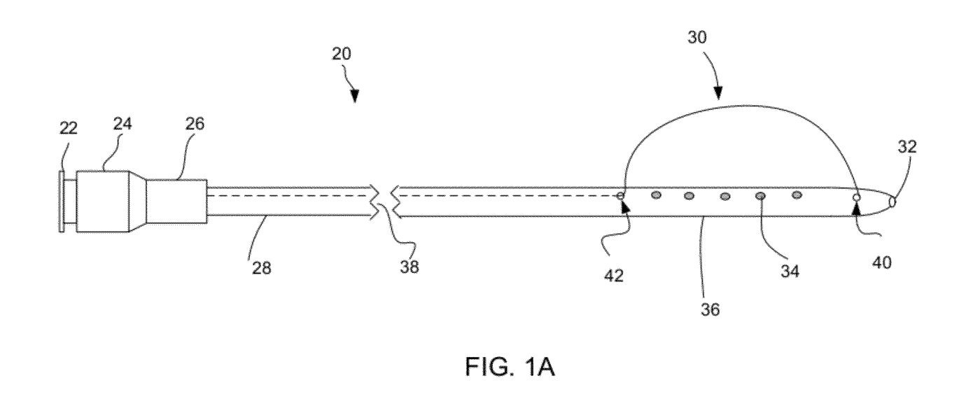

FIG. 1A depicts a catheter 20 comprising a flexible, elongate tube member 28 having a proximal end 22, a distal end 32, and a restraining portion 36. The wall of the restraining portion 36 toward the distal end 32 defines a series of drainage holes 34. The elongate tube member defines an internal lumen 38, which extends through the catheter and carries a flexible tension member 30, such as a suture thread. The tension member 30 extends through draw ports 40, 42 at two spaced positions on the restraining portion 36. The restraining portion 36 can be preformed into a “pigtail loop” shape from a shape-memory material or it can just extend along the longitudinal axis of the catheter. When the catheter is first introduced into the patient, a cannula can be inserted into the catheter lumen to help straighten the catheter. As shown in FIG. 1A, the restraining portion 36 extends along the horizontal axis. When the catheter reaches the drainage site, the cannula is removed, and the draw ports 40, 42 move toward each other. As a result, the restraining portion 36 is formed in the shape of a pigtail, as shown in FIG. 1B. The pigtail loop configuration can be helped into place and secured by manipulating the tension member 30 at the proximal end 22 of the catheter, where the hub 24 is located. After the desired pigtail is formed, the tension member 30 is locked into position via a hub-locking mechanism 26 to maintain the distal pigtail shape.

Other restraining means utilizing a preformed curve as an anchoring mechanism are possible, such as a malecot rib fixation. In such a configuration, longitudinal slits are located in the restraining portion of the catheter, so that a malecot rib comprising of multiple wings is formed as the tension member is manipulated at the proximal end.

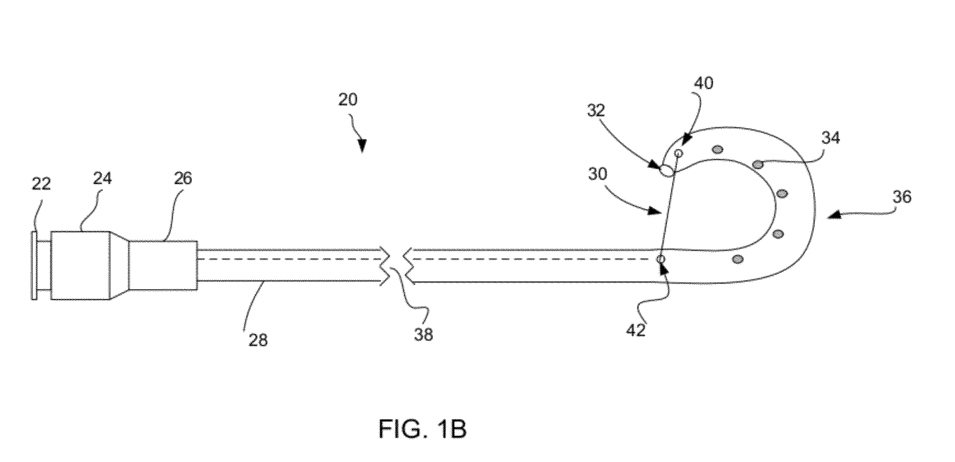

Referring to FIG. 2, an exemplary embodiment of an alternative anchoring mechanism is shown. Here, one or more anchoring members 250 are arranged circumferentially on a portion near the distal end 232 of the catheter shaft. Specifically, the restraining portion 236 of the catheter near the distal end 232 defines a spiral geometry on the exterior surface. In a preferred embodiment, the spiral geometric structure can be in the shape of a set of continuous protruding rims 250 that winds along the longitudinal axis of the catheter in a helical pattern. The catheter wall defines one or more drainage holes 234 that are longitudinally interspersed between at least a portion of the protruding rims 250. The set of protruding rims 250 is configured to make contact with human issue as the catheter is introduced into the patient's body, and the rims 250 can be locked into the anatomy via threading, rotating, linear indexing or similar action.

The spiral anchoring geometry can have more than one helical shape, with one or more series of rims that start and stop on different locations along the catheter 200. The pitch, or the distance from one point on the rim to a corresponding point on an adjacent rim measured parallel to the axis of the catheter, can vary. For example, the rims 250 can be spaced closer together at the proximal end, and farther apart at the distal end, or vice versa. This configuration can facilitate the interfacing of the rims 250 with different types of tissues encountered at various parts of the catheter. Additionally, the angles of the rims 250 relative to the longitudinal axis of the catheter can also vary. A difference in angle can affect the ease with which the rims are threaded to interlock with the patient's tissue, as well as the strength of the anchoring hold. The rims 250 themselves can vary in height, have different cross-sectional geometry (i.e., semi-circular, triangular, trapezoid) and be placed at one or more discrete locations along the catheter shaft.

The catheter 200 and/or anchoring geometry 250 can be constructed of thermoplastic polymers such as polyurethane, ethyl vinyl acetate (EVA), polyether block amide elastomer, polypropylene, or polyolefin elastomers. The catheter system can also be constructed of a thermoset plastic like silicone. The anchoring geometry 250 can be flexible or rigid in nature and can be different in material construction than the catheter shaft. The anchoring members 250 can be attached to the catheter via injection molding, tangential extrusion, RF welding, adhesives, or solvent bonding.

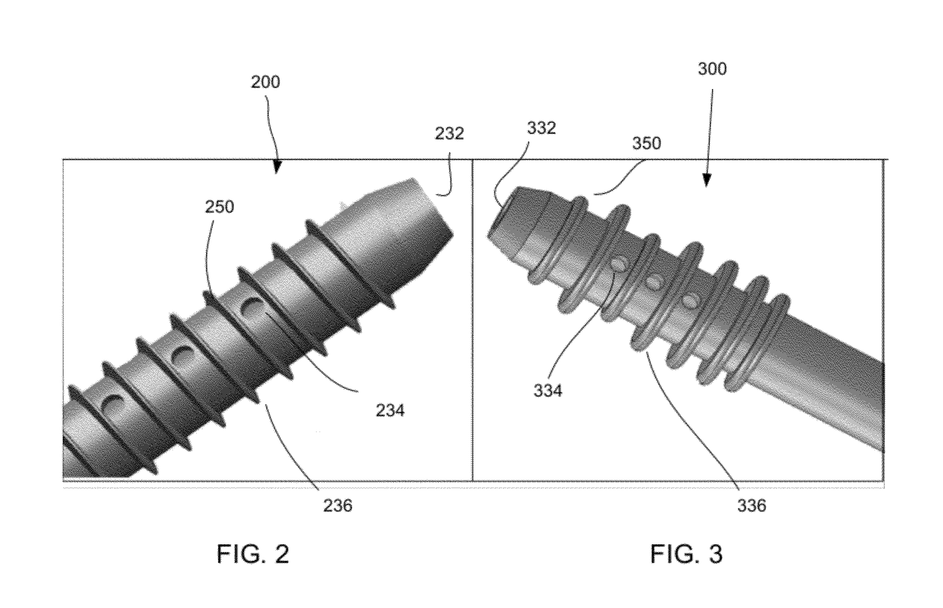

Referring to FIG. 3, another exemplary embodiment of the anchoring mechanism is shown. This embodiment includes annular rings 350 on the exterior of the catheter 300 along the restraining portion 336. Unlike the spiral anchoring geometry, the rims forming the annular rings 350 do not continue in a helical pattern along the length of the catheter. Rather, each ring 350 is located in spaced relation to another. As such, the interfacing of the rings 350 with the tissue of the patient would typically be effected by linear indexing, or similar action, rather than threading. Specifically, when the drainage site is reached, the catheter 300 can be linearly advanced one annular ring at a time in order to lock into the anatomy.

The rings 350 may be thick enough to appear on a monitoring apparatus such that the location of the rings 350 are apparent to the physician or technician who is manipulating the catheter. Alternatively, each ring 350 may comprise a radio opaque marker to facilitate the placement of the catheter. As the catheter may contain radio opaque fillers for x-ray visualization of the device, the rings themselves may be radio opaque markers.

The annular anchoring geometry can vary in height, and be positioned perpendicularly to the longitudinal axis of the catheter or at some bias/angle. The annular anchoring geometry can also have various cross-sectional geometry (i.e., semi-circular, triangular, trapezoid). Like the spiral anchoring geometry, the annular rings 350 can be placed at discrete locations along the catheter shaft, not just at the distal end of the catheter. Additionally, like the spiral anchoring geometry, the rings 350 can be located at multiple locations on the catheter to interlock with more than one location within the body of a patient.

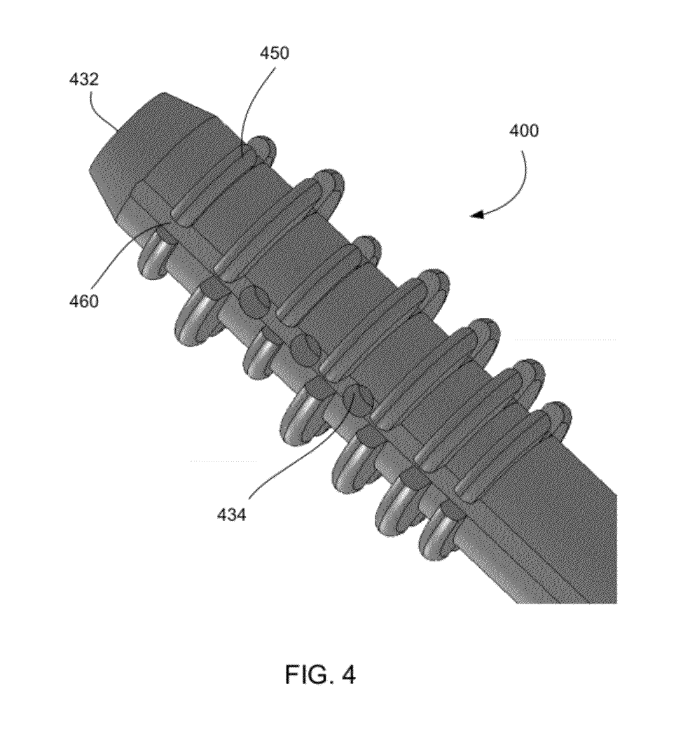

Referring to FIG. 4, an alternative embodiment of the anchoring geometries is shown. In this embodiment, the anchoring geometries in the form of annular rings 450 include slots 460 cut into them to enhance such properties as flexibility or promote anchoring. The slots can be formed in straight lines along the shaft of the catheter 400, as shown in FIG. 4. They can also be formed at an angle, intermittently, and/or in different patterns in order to facilitate the movement of the catheter and to enhance the interlocking ability with surrounding tissue. It is understood that although FIG. 4 only shows annular anchoring geometries, the concept relating to the inclusion of slots applies to other anchoring geometries as well.

The anchoring geometries mentioned in the foregoing discussion and shown above in FIGS. 2-4 can replace or supplement the traditional anchoring mechanism embodied by the pigtail loop shape or other shapes in the restraining portion of the catheter. Although not shown in the figures, the restraining portion of the catheter can include both a pigtail loop configuration and a spiral or annular anchoring geometry. In one embodiment, the pigtail loop configuration and the anchoring geometry can be combined such that the anchoring geometry overlaps with or spans the entire portion of the pigtail configuration. According to this embodiment, the spiral and annular anchoring geometries can be slotted so as to allow for the passage of the tension member such as a suture thread positioned along the exterior of the catheter between the draw ports. In an alternative embodiment according to the invention, the anchoring geometries can be placed in conjunction with the traditional anchoring mechanism at one or multiple locations without overlapping. For example, the pigtail loop configuration can be positioned at the distal end of the restraining portion, while one or more anchoring geometries can be positioned at the proximal end of the restraining portion. The pigtail loop configuration can also be positioned between two anchoring geometries.

The restraining portion as referenced herein spans one or more sections along the catheter that defines either a traditional anchoring mechanism (embodied by the pigtail loop configuration or the malecot rib configuration), one or more anchoring geometries arranged circumferentially on the shaft as disclosed herein (such as the spiral protruding rim or the annular rings), or a combination thereof. The length of the restraining portion may vary, according to the desired application. Typically, the restraining portion is located in the region medial to distal on the catheter, where the anchoring mechanism is to be activated in the body cavity. However, it is contemplated that the restraining portion can also be positioned closer to the proximal end of the catheter, as well as at multiple locations at any point between the proximal end and the distal end. In an exemplary embodiment, in addition to a first restraining portion comprising a pigtail configuration positioned near the distal end of the catheter, a second restraining portion comprising one or more anchoring geometries can be strategically positioned along the catheter between the proximal end and the first restraining portion, such that anchoring occurs at a tissue interface area in the body (e.g., at the skin of the patient).

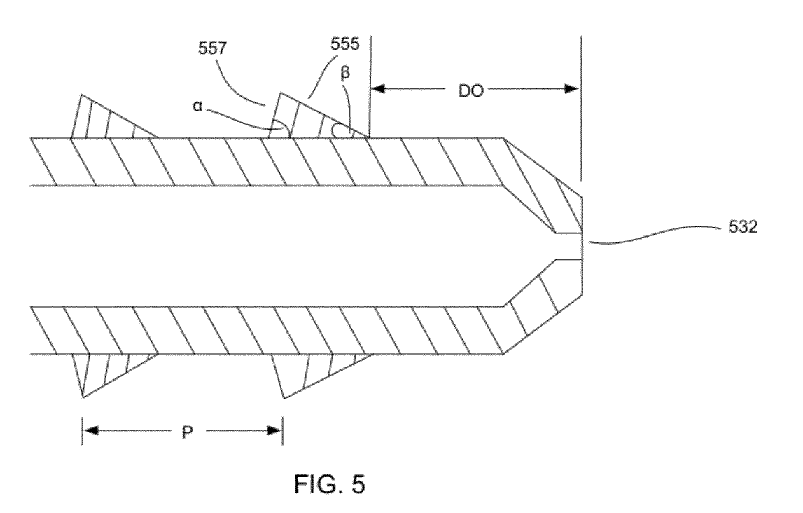

The shape of the rim(s) or the annual rings defining the anchoring geometries can vary (e.g., instead of having edges, they can be rounded). According to another exemplary embodiment, the anchoring geometries can comprise two opposing ramps, as shown in FIG. 5. In FIG. 5, a cross-sectional view of the anchoring geometries is shown at a distance DO (“distal offset”) from the distal end of the catheter. The distal ramp 555 can be set at the same or at a different angle as the proximal ramp 557 relative to the catheter axis. As illustrated in FIG. 5, α is the angle of the proximal ramp and β is the angle of the distal ramp. The steepness of the ramps can affect the ease with which the catheter enters the body of the patient and with which the catheter is removed. For example, if the ramp is more gradual at the distal end 532, then the insertion of the catheter will be easier. In contrast, if the ramp is more abrupt and has a flared shape at the distal end 532, then the insertion of the catheter will be more difficult. Similarly, if the ramp is more gradual at the proximal end, then the removal of the catheter will be easier. But if the proximal ramp is more abrupt, then it has a higher tendency to anchor the catheter in the body of the patient. In light of the desirability for ease of catheter insertion and a higher anchoring effect, the ramp angles can be designed accordingly. Also, FIG. 5 is only a schematic drawing showing an exemplary embodiment of the ramps, so the dimensions and the shapes of the ramps can vary in accordance to the invention. For example, the ramps may be curved instead of being flat, and the area at which the distal ramp and proximal ramp meet can be rounded instead of being a hard edge. The pitch (“P”), or the distance from one point on the anchoring geometry to a corresponding point on an adjacent anchoring geometry measured parallel to the axis of the catheter, can also vary.

Also contemplated herein are methods that can be performed using the subject devices or by other means. The methods can all comprise the act of providing a suitable device. Such provision can be performed by the end user. In other words, the “providing” merely requires the end user obtain, access, approach, position, set-up, activate, power-up or otherwise act to provide the requisite device in the subject method. Methods recited herein can be carried out in any order of the recited events which is logically possible, as well as in the recited order of events.

Exemplary embodiments, together with details regarding material selection and manufacture have been set forth above. As for other details of the presently described subject matter, these can be appreciated in connection with the above-referenced patents and publications as well as generally know or appreciated by those with skill in the art. The same can hold true with respect to method-based aspects in terms of additional acts as commonly or logically employed.

In addition, though the devices, systems and methods described herein have been presented herein in reference to exemplary embodiments, optionally incorporating various features, the devices, systems and methods described herein are not to be limited to that which is described or indicated as contemplated with respect to each variation. Various changes can be made to the subject matter described herein, and equivalents (whether recited herein or not included for the sake of some brevity) can be substituted without departing from the true spirit and scope of the disclosure.

Also, it is contemplated that any optional feature of the inventive variations described can be set forth and claimed independently, or in combination with any one or more of the features described herein. Stated otherwise, it is to be understood that each of the improvements described herein independently offer a valuable contributions to the state of the art. So too do the various other possible combination of the improvements/features described herein and/or incorporated by reference, any of which can be claimed.

Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Likewise, use of the term “typically” does not exclude other possibilities. It can indicate a preference, however, for the stated characteristic. Except as specifically defined herein, all technical and scientific terms used herein are to be given as broad a commonly understood meaning as possible while maintaining claim validity.

1. An indwelling percutaneous catheter comprising:
a central longitudinal catheter shaft;
one or more annular anchoring members arranged along an external portion of the catheter shaft.

2. The catheter of claim 1 further comprising a restraining portion, wherein said restraining portion of the catheter is preformed to have a pigtail structure.

3. The catheter of claim 1 further comprising a restraining portion, wherein said restraining portion of the catheter is preformed to have a malecot rib structure.

4. The catheter of claim 1, wherein the annular anchoring members are grouped in multiple clusters, each said cluster is located at a different location along the catheter shaft.

5. The catheter of claim 1, wherein one or more of the annular anchoring members do not traverse the full circumference of the catheter, leaving one or more gaps in said annular anchoring member or members.

6. The catheter of claim 1, wherein the annular anchoring members have varying heights.

7. The catheter of claim 1, wherein the annular anchoring members have varying pitches with respect to the longitudinal axis of the catheter shaft.

8. The catheter of claim 1, wherein the annular anchoring members have varying cross- sectional shape.

9. The catheter of claim 1, wherein the catheter shaft and the annular anchoring members are constructed of thermoplastic polymers.

10. The catheter of claim 1, wherein the annular anchoring members are attached to the catheter shaft through injection molding, tangential extrusion, RF welding, adhesives or solvent bonding.

11. The catheter of claim 1, wherein the annular anchoring members contain radio opaque fillers for x-ray visualization of the catheter.

12. A method of fixing a catheter within a body cavity comprising:
providing a catheter comprising an elongate tube member for insertion into the body cavity, the elongate tube member having a proximal end, a distal end, and a restraining portion, wherein the restraining portion includes an annular geometry on the external surface of the catheter shaft;
inserting said distal end of said elongate tube member into said body cavity;
securing the catheter to a tissue surface area with the body cavity by advancing the catheter and causing the restraining portion to lock with the tissue interface area.

13. The method of claim 12, the method further comprising the step of activating a pigtail structure found in the restraining portion.

14. The method of claim 12, the method further comprising the step of activating a malecot rib structure found in the restraining portion.

15. A catheter comprising an elongate tube member for insertion into a body cavity, the elongate tube member comprising:
a proximal end;
a distal end;
a first restraining portion defined by one or more annular anchoring members arranged along the external surface of the catheter shaft; and
a second restraining portion located between the first restraining portion and the distal end configured to form a pigtail curve.

16. A catheter comprising an elongate tube member for insertion into a body cavity, the elongate tube member comprising:
a proximal end;
a distal end;
a first restraining portion defined by one or more annular anchoring members arranged along the external surface of the catheter shaft; and
a second restraining portion located between the first restraining portion and the distal end configured to form a malecot rib structure.
